SEQUENCE LISTING (18 sequences)

SEQ ID NO: 1 - protein, 167 aa, Grifola frondosa
TYNGCSSSEQSALAAAASAAQSYVAESLSYLQTHTAATPRYTTWFGSYISSRHSTVLQHYTDMNSNDFSSYSFDCTCTAAGTFAYVYPNRFGTVYLCGAFWKAPTTGTDSQAGTLVHESSHFTRNGGTKDYAYGQAAAKSLATMDPDKAVMNADNHEYFSENNPAQS

SEQ ID NO: 2 - protein, 167 aa, Grifola frondosa
TYNGCSSSEQSALAAAASAAQSYVAESLSYLQTHTAATPRYTTWFGSYISSRHSTVLQHYTDMNSNDFSSYSFDCTCTAAGTFAYVYPNRFGTVYLCGAFWQAPTTGTDSQAGTLVHESSHFTRNGGTDDYAYGQAAAQSLATMNPDQAVMNADNHEYFSENNPAQS

SEQ ID NO: 3 - protein, 167 aa, Grifola frondosa; identical to SEQ ID NO: 2

SEQ ID NO: 4 - protein, 167 aa, Grifola frondosa
TYNGCSSSEQSALAAAASAAQSYVAESLSYLQTHTAATPRYTTWFGSYISSRHSTVLQHYTDMNSNDFSSYSFDCTCTAAGTFAYVYPNRFGTVYLCGAFWQAPTTGTDSQAGTLVHESSHFTRNGGTDDYAYGQAAAQSLATMNPDQAVMNANNHEYFSENNPAQS

SEQ ID NO: 5 - protein, 167 aa, Grifola frondosa
TYNGCSSSEQSALAAAASAAQSYVAESLSYLQTHTAATPRYTTWFGSYISSRHSTVLQHYTDMNSNDFSSYSFDCTCTAAGTFAYVYPNRFGTVYLCGAFWQAPTTGTDSQAGTLVHESSHFTRNGGTDDYAYGQAAAQSLATMNPDQAVMNANNHQYFSENNPAQS

SEQ ID NO: 6 - protein, 157 aa, Artificial Sequence
  IL-13 DARPin lysine substituted
ALDRRLLEAARAGQDDEVRILMANGADVNARDSYGSTPLHLAAREGHLEIVEVLLQYGADVNAADFIGDTPLHLAAYRGHLEIVEVLLQYGADVNASDITGETPLHLAAQIGHLEIVEVLLQHGADVNAQDIFGRTAFDISIDNGNEDLAEILQRAA

SEQ ID NO: 7 - protein, 124 aa, Artificial Sequence
  Albumin binding DARPin
DLGRRLLEAARAGQDDEVRILMANGADVNAADYFSHTPLHLAARNGHLEIVEVLLQYGADVNASDFAGRTPLHLAANDGHLEIVEVLLQHGADVNAQDIFGRTPADIAADNGHEDIAEVLQRAA

SEQ ID NO: 8 - protein, 124 aa, Artificial sequence
  albumin binding DARPin, single K
DLGRRLLEAARAGQDDEVRILMANGADVNAADYFSHTPLHLAARNGHLEIVEVLLQAGADVNASDFAGKTPLHLAANDGHLEIVEVLLQHGADVNAQDIFGRTPADIAADAGHEDIAEVLQRLN

SEQ ID NO: 9 - protein, 9 aa, Artificial sequence
  linker
AAAGGGGSG

SEQ ID NO: 10 - protein, 35 aa, Artificial sequence
  linker
GGGGSGGGGSGGGGSGGGGSGGGGSGGGGSGGGSS

SEQ ID NO: 11 - protein, 492 aa, Artificial sequence
  Artificial Construct
TYNGCSSSEQSALAAAASAAQSYVAESLSYLQTHTAATPRYTTWFGSYISSRHSTVLQHYTDMNSNDFSSYSFDCTCTAAGTFAYVYPNRFGTVYLCGAFWQAPTTGTDSQAGTLVHESSHFTRNGGTDDYAYGQAAAQSLATMNPDQAVMNADNHEYFSENNPAQSAAAGGGGSGDLGRRLLEAARAGQDDEVRILMANGADVNAADYFSHTPLHLAARNGHLEIVEVLLQYGADVNASDFAGRTPLHLAANDGHLEIVEVLLQHGADVNAQDIFGRTPADIAADNGHEDIAEVLQRAAGGGGSGGGGSGGGGSGGGGSGGGGSGGGGSGGGSSALDRRLLEAARAGQDDEVRILMANGADVNARDSYGSTPLHLAAREGHLEIVEVLLQYGADVNAADFIGDTPLHLAAYRGHLEIVEVLLQYGADVNASDITGETPLHLAAQIGHLEIVEVLLQHGADVNAQDIFGRTAFDISIDNGNEDLAEILQRAA

SEQ ID NO: 12 - protein, 424 aa, Artificial sequence
  Artificial Construct
SSYSFDCTCTAAGTFAYVYPNRFGTVYLCGAFWQAPTTGTDSQAGTLVHESSHFTRNGGTDDYAYGQAAAQSLATMNPDQAVMNANNHEYFSENNPAQSAAAGGGGSGDLGRRLLEAARAGQDDEVRILMANGADVNAADYFSHTPLHLAARNGHLEIVEVLLQYGADVNASDFAGRTPLHLAANDGHLEIVEVLLQHGADVNAQDIFGRTPADIAADNGHEDIAEVLQRAAGGGGSGGGGSGGGGSGGGGSGGGGSGGGGSGGGSSALDRRLLEAARAGQDDEVRILMANGADVNARDSYGSTPLHLAAREGHLEIVEVLLQYGADVNAADFIGDTPLHLAAYRGHLEIVEVLLQYGADVNASDITGETPLHLAAQIGHLEIVEVLLQHGADVNAQDIFGRTAFDISIDNGNEDLAEILQRAA

SEQ ID NO: 13 - protein, 492 aa, Artificial sequence
  Artificial Construct
TYNGCSSSEQSALAAAASAAQSYVAESLSYLQTHTAATPRYTTWFGSYISSRHSTVLQHYTDMNSNDFSSYSFDCTCTAAGTFAYVYPNRFGTVYLCGAFWQAPTTGTDSQAGTLVHESSHFTRNGGTDDYAYGQAAAQSLATMNPDQAVMNADNHQYFSENNPAQSAAAGGGGSGDLGRRLLEAARAGQDDEVRILMANGADVNAADYFSHTPLHLAARNGHLEIVEVLLQYGADVNASDFAGRTPLHLAANDGHLEIVEVLLQHGADVNAQDIFGRTPADIAADNGHEDIAEVLQRAAGGGGSGGGGSGGGGSGGGGSGGGGSGGGGSGGGSSALDRRLLEAARAGQDDEVRILMANGADVNARDSYGSTPLHLAAREGHLEIVEVLLQYGADVNAADFIGDTPLHLAAYRGHLEIVEVLLQYGADVNASDITGETPLHLAAQIGHLEIVEVLLQHGADVNAQDIFGRTAFDISIDNGNEDLAEILQRAA

SEQ ID NO: 14 - protein, 492 aa, Artificial sequence
  Artificial Construct
TYNGCSSSEQSALAAAASAAQSYVAESLSYLQTHTAATPRYTTWFGSYISSRHSTVLQHYTDMNSNDFSSYSFDCTCTAAGTFAYVYPNRFGTVYLCGAFWQAPTTGTDSQAGTLVHESSHFTRNGGTDDYAYGQAAAQSLATMNPDQAVMNANNHQYFSENNPAQSAAAGGGGSGDLGRRLLEAARAGQDDEVRILMANGADVNAADYFSHTPLHLAARNGHLEIVEVLLQYGADVNASDFAGRTPLHLAANDGHLEIVEVLLQHGADVNAQDIFGRTPADIAADNGHEDIAEVLQRAAGGGGSGGGGSGGGGSGGGGSGGGGSGGGGSGGGSSALDRRLLEAARAGQDDEVRILMANGADVNARDSYGSTPLHLAAREGHLEIVEVLLQYGADVNAADFIGDTPLHLAAYRGHLEIVEVLLQYGADVNASDITGETPLHLAAQIGHLEIVEVLLQHGADVNAQDIFGRTAFDISIDNGNEDLAEILQRAA

SEQ ID NO: 15 - protein, 491 aa, Artificial sequence
  Artificial Construct
TYNGCSSSEQSALAAAASAAQSYVAESLSYLQTHTAATPRYTTWFGSYISSRHSTVLQHYTDMNSNDFSSYSFDCTCTAAGTFAYVYPNRFGTVYLCGAFWQAPTTGTDSQAGTLVHESSHFTRNGGTDDYAYGQAAAQSLATMNPDQAVMNADNHEYFSENNPAQSAAAGGGGSGDLGRRLLEAARAGQDDEVRILMANGADVNAADYFSHTPLHLAARNGHLEIVEVLLQAGADVNASDFAGKTPLHLAANDGHLEIVEVLLQHGADVNAQDIFGRTPADIAADAGHEDIAEVLQRLNGGGGSGGGGSGGGGSGGGGSGGGGSGGGGSGGGSSGLDRRLLEAARAGQDDEVRILMANGADVNATDEFDSTPLHLAARHGHLEIVEVLLQYGADVNAADFIGDTPLHLAAYRGHLEIVEVLLQYGVDVNADDHGDTPLHLAASTGHLEIVEVLLQHGADVNAQDIFGRTAFDISIDNGNEDLAEILQRAA

SEQ ID NO: 16 - protein, 491 aa, Artificial sequence
  Artificial Construct
TYNGCSSSEQSALAAAASAAQSYVAESLSYLQTHTAATPRYTTWFGSYISSRHSTVLQHYTDMNSNDFSSYSFDCTCTAAGTFAYVYPNRFGTVYLCGAFWQAPTTGTDSQAGTLVHESSHFTRNGGTDDYAYGQAAAQSLATMNPDQAVMNANNHEYFSENNPAQSAAAGGGGSGDLGRRLLEAARAGQDDEVRILMANGADVNAADYFSHTPLHLAARNGHLEIVEVLLQAGADVNASDFAGKTPLHLAANDGHLEIVEVLLQHGADVNAQDIFGRTPADIAADAGHEDIAEVLQRLNGGGGSGGGGSGGGGSGGGGSGGGGSGGGGSGGGSSGLDRRLLEAARAGQDDEVRILMANGADVNATDEFDSTPLHLAARHGHLEIVEVLLQYGADVNAADFIGDTPLHLAAYRGHLEIVEVLLQYGVDVNADDHGDTPLHLAASTGHLEIVEVLLQHGADVNAQDIFGRTAFDISIDNGNEDLAEILQRAA

SEQ ID NO: 17 - protein, 491 aa, Artificial sequence
  Artificial Construct
TYNGCSSSEQSALAAAASAAQSYVAESLSYLQTHTAATPRYTTWFGSYISSRHSTVLQHYTDMNSNDFSSYSFDCTCTAAGTFAYVYPNRFGTVYLCGAFWQAPTTGTDSQAGTLVHESSHFTRNGGTDDYAYGQAAAQSLATMNPDQAVMNADNHQYFSENNPAQSAAAGGGGSGDLGRRLLEAARAGQDDEVRILMANGADVNAADYFSHTPLHLAARNGHLEIVEVLLQAGADVNASDFAGKTPLHLAANDGHLEIVEVLLQHGADVNAQDIFGRTPADIAADAGHEDIAEVLQRLNGGGGSGGGGSGGGGSGGGGSGGGGSGGGGSGGGSSGLDRRLLEAARAGQDDEVRILMANGADVNATDEFDSTPLHLAARHGHLEIVEVLLQYGADVNAADFIGDTPLHLAAYRGHLEIVEVLLQYGVDVNADDHGDTPLHLAASTGHLEIVEVLLQHGADVNAQDIFGRTAFDISIDNGNEDLAEILQRAA

SEQ ID NO: 18 - protein, 492 aa, Artificial Sequence
  Artificial construct
ATYNGCSSSEQSALAAAASAAQSYVAESLSYLQTHTAATPRYTTWFGSYISSRHSTVLQHYTDMNSNDFSSYSFDCTCTAAGTFAYVYPNRFGTVYLCGAFWQAPTTGTDSQAGTLVHESSHFTRNGGTDDYAYGQAAAQSLATMNPDQAVMNANNHQYFSENNPAQSAAAGGGGSGDLGRRLLEAARAGQDDEVRILMANGADVNAADYFSHTPLHLAARNGHLEIVEVLLQAGADVNASDFAGKTPLHLAANDGHLEIVEVLLQHGADVNAQDIFGRTPADIAADAGHEDIAEVLQRLNGGGGSGGGGSGGGGSGGGGSGGGGSGGGGSGGGSSGLDRRLLEAARAGQDDEVRILMANGADVNATDEFDSTPLHLAARHGHLEIVEVLLQYGADVNAADFIGDTPLHLAAYRGHLEIVEVLLQYGVDVNADDHGDTPLHLAASTGHLEIVEVLLQHGADVNAQDIFGRTAFDISIDNGNEDLAEILQRAA

(19) United States
(12) Patent Application Publication
(10) Pub. No.: US 2019/0343963 A1
(43) Pub. Date: Nov. 14, 2019

(54) CONJUGATED PROTEASE TARGETING MOIETIES

(71) Applicant: MEDIMMUNE LIMITED, CAMBRIDGE (GB)

(72) Inventors: Carole Urbach, Cambridge (GB); Nathaniel Gordon, Cambridge (GB)

(21) Appl. No.: 16/095,031

(22) PCT Filed: Apr. 21, 2017
(86) PCT No.: PCT/EP2017/059550

§ 371 Date: Oct. 19, 2018

Related U.S. Application Data
(60) Provisional application No. 62/326,220, filed on Apr. 22, 2016.

(51) Int. Cl. A61K 47/68; A61K 47/64; A61K 38/48

(52) U.S. Cl. CPC A61K 47/6815; A61K 38/4886; A61K 47/64

(57) ABSTRACT

A protease therapeutic comprising a Lysine-specific metalloprotease domain conjugated to a first targeting moiety.

The present invention relates to proteases conjugated to targeting moieties and in particular to therapeutic targeting moieties.

Targeted therapeutics such as antibodies, antibody domains, receptor domains, and other types of antigen binding domains are the types of therapeutic molecules currently used to specifically neutralize a target antigen. They rely for therapeutic effect on stoichiometric, high-affinity, non-covalent, reversible inhibition of their target antigen. The limitation associated with this is that high and potentially unsafe or impractical doses can be required, e.g. for abundant and/or rapidly-cleared targets. In addition, they may have poor distribution in a tumour or tissue.

For this reason, other potential mechanisms and therapeutic molecules have been sought out. One possibility that has been investigated is protease therapeutics. Such proteases catalyse hydrolysis of peptide bonds, which is effectively irreversible, and as such proteases offer the potential for several clinical advantages. For example, a protease therapeutic will irreversibly neutralise a target by hydrolysis of covalent associations and thus the total amount of a soluble antigen in circulation will not increase due to sequestration, as can be the case with antibodies and other binding domains. As a protease has an irreversible mechanism of action, it may not be subject to many of the limitations of stoichiometric therapies, such as neutralising antibodies, antibody mimetics, and related classes of therapeutics. This would lead to significantly lower dosing, particularly for abundant and/or short half-life antigens, and potentially better pharmacokinetics and biodistribution.

One limitation of such protease therapeutics is that no protease generally exists with sufficient target specificity to serve as a viable therapeutic agent. In particular, biotherapeutic engineering of proteases is not routine and not always achievable; there are no de novo means to engineer the specificity of these molecules, in contrast with the biotherapeutic engineering of antibodies, antibody fragments, antibody mimetics and related binding domains, which may be easily engineered for biophysical and biochemical properties which make them suitable for therapeutic applications, and there are a number of routine techniques for the de novo discovery of binding domains specific for a given therapeutic target.

Another limitation of protease therapeutics is the potential for interaction, inhibition, and clearance by endogenous human serum protease inhibitors. Naturally occurring protease inhibitors found in the human body represent a critical component impinging on the therapeutic potential of protease therapeutics and is well established for conventional proteases. In particular proteolytic reaction with the high-concentration, irreversible, serum protease inhibitors—the serpins and alpha-2-macroglobulin—leads to the rapid inhibition and rapid clearance of reactive proteases and protease therapeutics.

Serpins are a class of serine protease inhibitors, many of which are amongst the highest concentration polypeptides in human serum. These inhibitors present a reactive loop which is a substrate for a wide variety of proteases. If a serine protease reacts with residues in the reactive loop, the catalytic cycle is interrupted, trapping a covalent protease-serpin complex via a gross conformational change in the serpin itself. This complex is recognised by cell surface receptors through the new conformation induced in the serpin and is rapidly cleared from serum. Any therapeutic serine protease and many cysteine proteases may be susceptible to serpin induced clearance from systemic circulation after administration, severely limiting their half-life.

Alpha-2-macroglobulin (a2M) is a large (720 kDa) tetrameric, pan specific protease inhibitor and is a part of the innate immune system. It exists at high concentration (approximately 10 μM) in circulation, and is part of the body's defence against unregulated or foreign proteases. It has a unique mechanism of action: after being triggered by hydrolysis of an unstructured bait-region—a ready substrate for proteases from across mechanistic classes—a reactive thioester in a2M is exposed which reacts with solvent exposed residues in the protease, forming a covalent linkage between a2M and the protease. Additionally, alpha-2-macroglobulin undergoes a large conformational change whereby the protease is trapped within a ‘cage’ formed by a2M. This sequesters the protease away from macromolecular substrates, preventing it from hydrolysing proteins in circulation. Furthermore, the conformational change also exposes sites on a2M recognised with high affinity by cell surface receptors on hepatocytes, and the complex is rapidly cleared from circulation. Reaction with alpha-2-macroglobulin constitutes the primary mechanism by which existing protease therapeutics are eliminated after systemic administration.

Reaction with serpins and alpha-2-macroglobulin would therefore greatly accelerate their clearance, with a concomitant decrease in exposure and pharmacodynamics effects. As such, protease therapeutics are currently excluded from many therapeutic applications.

There is a need for protease therapeutics for proteolytic therapy which avoid such inhibitors. There is a need for protease therapeutics for proteolytic therapy which can have a suitable target specificity. There is a need for protease therapeutics for proteolytic therapy which have potential for long serum half-life by e.g. escaping serpins and a2M.

### BRIEF SUMMARY

The present invention meets one or more of the above needs by providing a protease therapeutic comprising a lysine-specific metalloprotease domain conjugated to a first targeting moiety.

In some aspects the lysine-specific metalloprotease domains are metalloendoproteases, optionally selected from the M35 family. For instance a Grifola frondosa metalloendoprotease (GfMEP) domain.

In some aspects some or all of the lysine residues within the protease therapeutic have been deleted and/or substituted.

In some aspects the protease therapeutics have been modified to reduce the proteolytic activity of the metalloprotease domain, preferably whilst maintaining the specificity of the metalloprotease domain.

In some aspects the first targeting moiety is selected from the group consisting of a targeting peptide, an antibody mimetic, a Tn3 scaffold, an antibody or antigen binding fragment thereof, a scFv, a Fab, a Fab′, a domain antibody, a DARPin, an aptamer and a receptor domain. A second targeting moiety may also be incorporated in the protease therapeutic to confer bispecific binding against two targets or two epitopes on the same target.

In some aspects the protease therapeutic comprises a second moiety to extend the half-life of the protease therapeutic.

In some aspects, the protease therapeutic is expressed as a fusion construct. In other aspects the metalloprotease domain and targeting moieties are expressed separately and chemically conjugated

The protease therapeutics disclosed herein are of particular use in therapy.

### BRIEF DESCRIPTION OF THE FIGURES

FIGS. 1A and 1B shows a schematic of a protease therapeutic according to the invention and its mechanism of action.

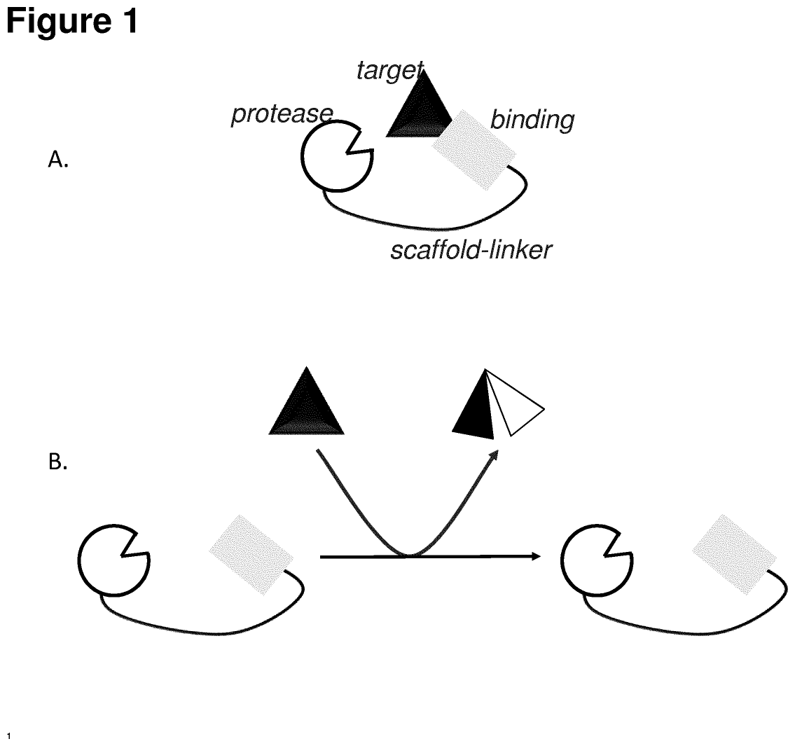

FIG. 2 shows the relative proteolytic activities of MMP-8, GfMEP and trypsin. Notably GfMEP proteolysis a wider range of substrates than MMP-8.

FIG. 3 shows the relative activity of GfMEP to thermolysin in the presence of alpha-2-macroglobulin.

FIG. 4 shows a ribbon diagram of GfMEP.

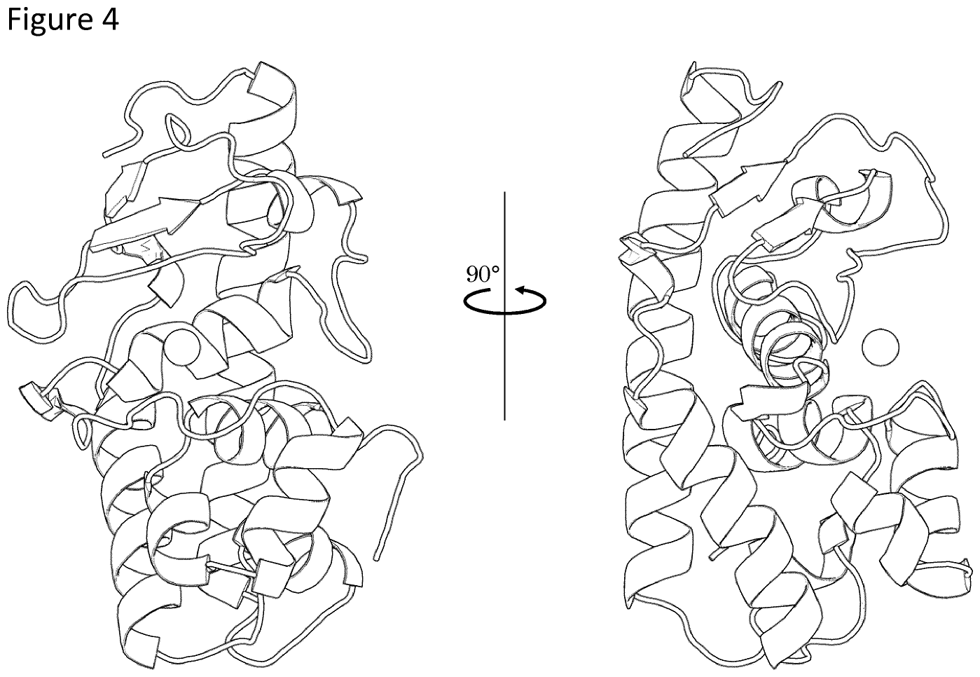

FIG. 5 shows a ribbon diagram of the active site of GfMEP with key residues labelled.

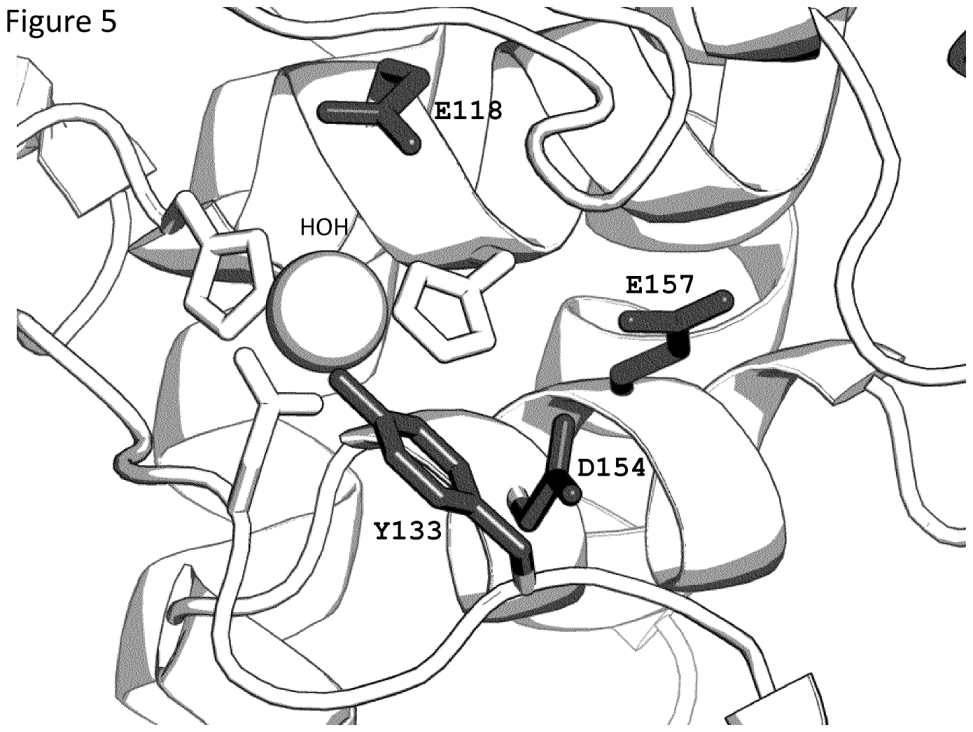

FIG. 6 shows the relative activity of wild type, D154N, E157Q and D154n/E157Q mutant metalloendoproteases.

FIG. 7 shows the lysine specificity of wild type, D154N, E157Q and D154N/E157Q (NQ) mutant metalloendoproteases.

FIG. 8 shows the inhibition of II-13 using CAT-354 (an IgG1 anti-IL-13 antibody), a CAT-354 fab and protease therapeutics according to the present invention.

FIG. 9 shows the inhibition of II-13 using CAT-354 and protease therapeutics comprising albumin binding domains according to the present invention.

FIG. 10 shows eosinophil levels in an ova-induced air pouch lavage model.

### SEQUENCES

The following sequences are provided:

SEQ ID NO: 1 Wild-type GfMEP domain

SEQ ID NO: 2 Wild-type GfMEP domain, lysine residues substituted

SEQ ID NO: 3 Wild-type GfMEP domain, lysine residues substituted and Aspartic Acid substituted for Asparagine at position 154

SEQ ID NO: 4 Wild-type GfMEP domain, lysine residues substituted and Glutamic Acid substituted for Glutamine at position 157

SEQ ID NO: 5 Wild-type GfMEP domain, lysine residues substituted, Aspartic Acid substituted for Asparagine at position 154 and Glutamic Acid substituted for Glutamine at position 157

SEQ ID NO: 6 IL-13-binding DARPin, lysine residues substituted

SEQ ID NO: 7 Albumin-binding DARPin

SEQ ID NO: 8 Albumin-binding DARPin, all but one lysine residues substituted

SEQ ID NO: 9 First linker sequence

SEQ ID NO: 10 Second linker sequence

SEQ ID NO: 11 Protease therapeutic (SH111 wt del K)

SEQ ID NO: 12 Protease therapeutic (SH111 D154N del K)

SEQ ID NO: 13 Protease therapeutic (SH111 E157Q del K)

SEQ ID NO: 14 Protease therapeutic (SH111 D154N E157Q (NQ) del K)

SEQ ID NO: 15 Protease therapeutic (SH111 wt 7g11 a1b22 single K)

SEQ ID NO: 16 Protease therapeutic (SH111 D154N 7g11 a1b22 single K)

SEQ ID NO: 17 Protease therapeutic (SH111 E157Q 7g11 a1b22 single K)

SEQ ID NO: 18 Protease therapeutic (SH111 D154N E157Q (NQ) 7g11 a1b22 single K)

### DETAILED DESCRIPTION

The present inventors have surprisingly found that, through the choice of a lysine specific metalloprotease domain they have been able to generate protease therapeutics that overcome the challenges facing previously described protease therapeutics (such as antibody-enzyme constructs of EP patent application EP 1730198). The lysine specific metalloprotease domains used in the present invention are advantageously both more promiscuous than previously described as part of a protease therapeutic (i.e. they can target a wider variety of substrates) and less subject to inhibition by naturally occurring protease inhibitory mechanisms. The lysine specific metalloprotease domains used in the present invention are (i) not susceptible to inhibition by serine protease inhibitors (SERPINS) and (ii) not subject to inhibition by alpha-2-macroglobulin as alpha-2-macroglobulin does not contain lysine residues within its bait region, which must first be cleaved before it can have an inhibitory effect.

In some aspects the lysine specific metalloprotease domains comprise metalloendoproteases. The metalloendoproteases may be selected from the M35 family. In a particular the Grifola frondosa metalloendoprotease (GfMEP) may be used.

In some aspects the metalloprotease protease domain is modified such that it is a non-naturally occurring mutant metalloprotease domain.

The metalloprotease protease domain may be modified to remove all protease accessible lysine residues such that it is not susceptible to autocatalysis. The protease accessibility of a given lysine residue can be assessed on the basis of the tertiary structure of the metalloprotease, for instance surface exposed lysines are more likely to be protease accessible, or can be assessed experimentally by incubating the metalloprotease by itself and then running the incubated metalloprotease on a gel to see if more than one band is present, indicating cleavage has occurred.

The metalloprotease domain may be modified such that it contains no primary amines, except for the N-terminal amine. Such modification prevents activated alpha-2-macroglobulin binding to the metalloprotease through the formation of a thioester bond and inactivating the metalloprotease

In some aspects, the metalloprotease domain has been modified to reduce the proteolytic activity of the metalloprotease domain. Such modifications can be made by modifying key residues in the active site of the metalloprotease. The present invention provides examples of modifications made to GfMEP, but it will be apparent to the person skilled in the art how other metalloprotease domains could be similarly modified. For instance by aligning the metalloprotease sequences with GfMEP as shown below, one can identify the residues at positions equivalent to 118, 133, 154 and 157 of the GfMEP set forth in SEQ ID NO. 1.

TABLE 1

| | |
|---|---|
| | 1 10 20 30 40 |
| | \| \| \| \| \| |
| GfMEP/1-167 | TYNGCSSSEQ SALAAAASAA QSYVAESLSY LQTHTAATPR YTTWFGSYIS |
| PoMEP/1-167 | TFVGCSATRQ TQLNAAASQA QTYAANALSY LNSHTSSTTR YTTWFGTFVT |
| AmMEP/1-167 | SYNGCTSSRQ TTLVSAAAAA QTYAQSSYNY LSSHTASTTR YVTWFGPYTS |
| | 50 60 70 80 90 |
| | \| \| \| \| \| |
| GfMEP/1-167 | SRHSTVLQHY TDMNSNDFSS YSFDCTCTAA GTFAYVYPNR FGTVYLCGAF |
| PoMEP/1-167 | SKYNTVLSHF SSISSNTFSS YTFDCTCSDS GTYAFVNPSN FGYVTLCGAF |
| AmMEP/1-167 | ARHSTVLSCF SNMLAYPYAN YEYDCTCTES DVYAYVYPSQ FGTIYLCGAF |
| | 100 110 120 130 140 |
| | \| \| \| \| \| |
| GfMEP/1-167 | WKAPTTGTDS QAGTLVHESS HFTRNGGTKD YAYGQAAAKS LATMDPDKAV |
| PoMEP/1-167 | WNAPVAGTDS RGGTLIHESS HFTRNGGTDD HVYGQAGAQS LARSNPAQAI |
| AmMEP/1-167 | WQTTTTGTDS RGGTLIHESS HFTIICGTQD YAYGQSAAKS LASSNPSEAI |
| | 150 160 |
| | \| \| |
| GfMEP/1-167 | MNADNHEYFS ENNPAQS |
| PoMEP/1-167 | DNADSHEYFA ENNPALA |
| AmMEP/1-167 | KNADNYEYFA ENNPAQS |

Without wishing to be bound by theory reducing the proteolytic activity of the metalloprotease domain, increases the specificity of the protease therapeutic by reducing off target activity whilst preserving proteolytic activity of the target bound by the targeting moiety. This reduction in off target activity relative to on target activity may be referred to as an improved therapeutic index or increased therapeutic window.

In some aspects the modifications to the metalloprotease domain maintain the specificity of the metalloprotease domain such that they still proteolyse the target of the protease therapeutic.

Suitable modifications to reduce the proteolytic activity of the protease therapeutic whilst maintaining specificity comprise modification of one or more residues of the metalloendoprotease domain equivalent to residues 118, 133, 154 and 157 of SEQ ID NO. 1. For example one or more residues selected from the group of 118, 133, 154 and 157 of a GfMEP domain having SEQ ID NO. 1 may be substituted. Suitable substitutions may be selected from the group consisting of E118D, E118Q, E118N, E1185, E118A, Y133F, D154N, and E157Q.

It will be appreciated that other modifications may be made to the metalloprotease domains disclosed herein without comprising the functionality of the metalloprotease domain and such GfMEP protease domains may comprise a sequence having at least 90%, at least 95%, at least 98% or at least 99% identity to SEQ ID NO: 1.

Particular embodiments of GfMEP domains that are suitable for incorporation in a protease therapeutics according to the present invention may comprise a sequence selected from the group consisting of SEQ ID NO.s 2 to 5.

In an embodiment the GfMEP protease domain comprises SEQ ID NO: 2.

In an embodiment the GfMEP protease domain comprises SEQ ID NO. 3

In an embodiment the GfMEP protease domain comprises SEQ ID NO. 4

In an embodiment the GfMEP protease domain comprises SEQ ID NO. 5

In other embodiments the GfMEP protease domain, may further comprise a substitution at position 118, such as E118D, E118Q, E118N, E118S or E118A.

In other embodiments the GfMEP protease domain, may further comprise a substitution at position 133, such as Y133F.

The person skilled in the art will appreciate that any of the substitutions above may be made alone or in combination with substitutions at one, two or three of the other recited positions.

In one embodiment the GfMEP protease domain comprises the sequence of SEQ ID NO: 1.

In some aspects, the first targeting moiety of the protease therapeutic is selected from the group consisting of a targeting peptide, an antibody mimetic, a Tn3 scaffold, an antibody or antigen binding fragment thereof, a scFv, a Fab, a Fab′, a domain antibody, a DARPin, an aptamer and a receptor domain.

In an aspect the first targeting moiety is an antibody, or antigen binding fragment thereof.

In an aspect the first targeting moiety is a DARPin.

In some aspects the protease therapeutic is further conjugated to a second moiety. Such second moieties can be second targeting moieties such as a targeting peptide, an antibody mimetic, a Tn3 scaffold, an antibody or antigen binding fragment thereof, a scFv, a Fab, Fab′, a domain antibody, a DARPin, an aptamer or a receptor domain. The first and second targeting moieties may be directly conjugated so as to form a bispecific targeting moiety, which binds two independent targets or two epitopes on the same target.

In other aspects the second moiety may be a half-life extension moiety. Such moieties may be selected from the group consisting of an albumin binding domain, albumin, an Fc region, polyethylene glycol, a XTEN fusion peptide, and a Proline/Alanine/Serine (PAS) polypeptide. For instance, the albumin binding domain is an albumin-binding DARPin. Such half-life extended constructs may advantageously increase the overall level of inhibition over a longer period compared to protease therapeutics without extended half-lives.

In an embodiment the first targeting molecule is an anti-II-13 DARPin.

In some aspects the metalloprotease domain is conjugated to the first targeting moiety via a first linker.

In some aspects the second targeting moiety is conjugated to the protease therapeutic via a second linker.

In some aspects the targeting moieties, half-life extension moieties and/or the linkers are free of protease accessible lysine residues or entirely lysine free, and as such are not subject to autocatalysis.

In an embodiment the protease therapeutic comprises a sequence according to SEQ ID NO: 11.

In an embodiment the protease therapeutic comprises a sequence according to SEQ ID NO: 12.

In an embodiment the protease therapeutic comprises a sequence according to SEQ ID NO: 13.

In an embodiment the protease therapeutic comprises a sequence according to SEQ ID NO: 14.

In an embodiment the protease therapeutic comprises a sequence according to SEQ ID NO: 15.

In an embodiment the protease therapeutic comprises a sequence according to SEQ ID NO: 16.

In an embodiment the protease therapeutic comprises a sequence according to SEQ ID NO: 17.

In an embodiment the protease therapeutic comprises a sequence according to SEQ ID NO: 18.

In some aspects the protease therapeutic may be expressed as a recombinant fusion peptide or protein. Any suitable method known in the art can be used to express and purify the recombinant fusion peptide or protein. Exemplary methods for expressing and purifying the protease therapeutic are described in Example 5.

In some aspects the metalloprotease domain and targeting moieties may be expressed separately and chemically conjugated. Suitable methods of chemical conjugation include, but are not limited to solid phase chemical ligation, cysteine-maleimide conjugation, oxime conjugation, or click chemistry conjugation.

In an aspect the protease therapeutics disclosed herein are suitable for use in therapy. The protease therapeutics may be useful in the treatment of cancer, a respiratory condition, an inflammatory condition, cardiovascular condition or metabolic condition. As such, disclosed herein are methods of treatment comprising administering a therapeutically effective amount of the protease therapeutic disclosed herein to a patient in need of therapy. Such methods of treatment may be administered where the patient has cancer, a respiratory condition, an inflammatory condition, a cardiovascular condition or a metabolic condition.

### EXAMPLES

### Example 1—Alpha-2-macroglobulin Assay

Alpha-2-macrglobulin was diluted from 4 μM to 0 μM in assay buffer (PBS containing 1mM CaCl2 and 100 μM ZnCl2). Separately, solutions of proteases were prepared at 500 nM (thermolysin) and 100 nM (GfMEP) were prepared in assay buffer. The protease dilutions and macroglobulin dilutions were then mixed at 1:1 ratios, and incubated at 37° C. for 30 min.

A macromolecular YFP-CFP labelled FRET substrate was prepared at approximately 2 μM in assay buffer. Ten microliters of this solution was aliquoted to wells of a 384 well black bottom fluorescent plate.

Following the 30 min 37° C. incubation, 10 μL of protease:macroglobulin samples were added to substrate containing wells of the 96 well black bottom fluorescent plate. The plates were immediately transferred to an Envision fluorescent plate reader and assayed for fluorescence six times at 3 min intervals with excitation wavelength set to λexc=414 nm emission wavelengths read at λem=475 nm and λem=527 nm. Hydrolysis of the substrate was then determined by changes to the ratio of fluorescence measured in these emission bands as a function of time (as follows).

The ratio of λem=527 nm to λem=475 was plotted as a function of time. This was then fit by a single exponential to obtain half-life (kobs) for the reaction. The activity (arbitrary units, proportional to kcat/KM) was calculated from the observed decay rate using a form of the integrated rate equation: activity ∝ (kobs×[E]) for the limiting behaviour [S]«KM. This ‘progress curve fitting’ was applied to all samples to determine their residual activity after incubation with alpha-2-macroglobulin.

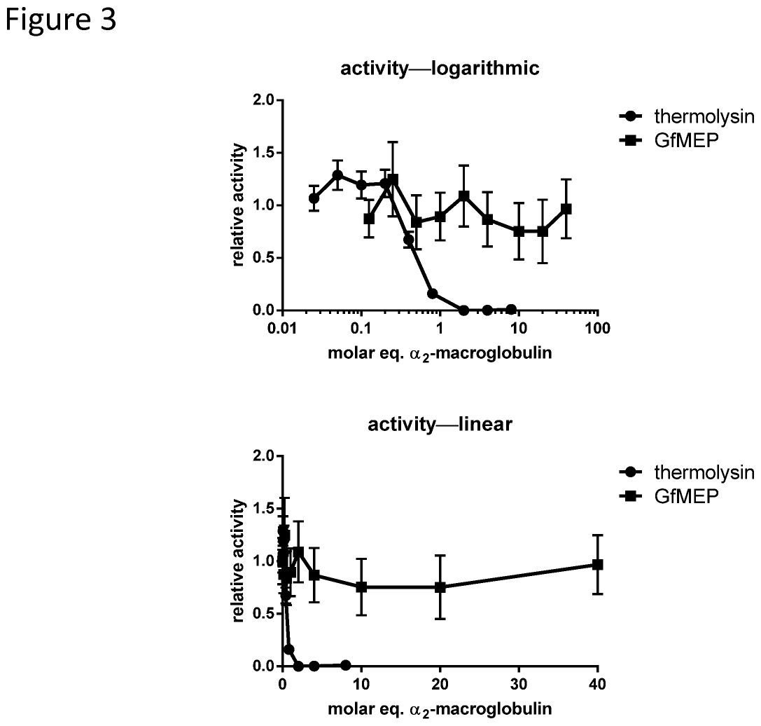

The relative activity in each of the samples was determined by normalizing by the activity in the samples containing no alpha-2-macroglobulin. The relative activity thus measured was plotted as a function of the equivalents of alpha-2-macroglobulin (either logarithmic or linear graphs). This is depicted in FIG. 3.

### Example 2—Hydrolysis Assay

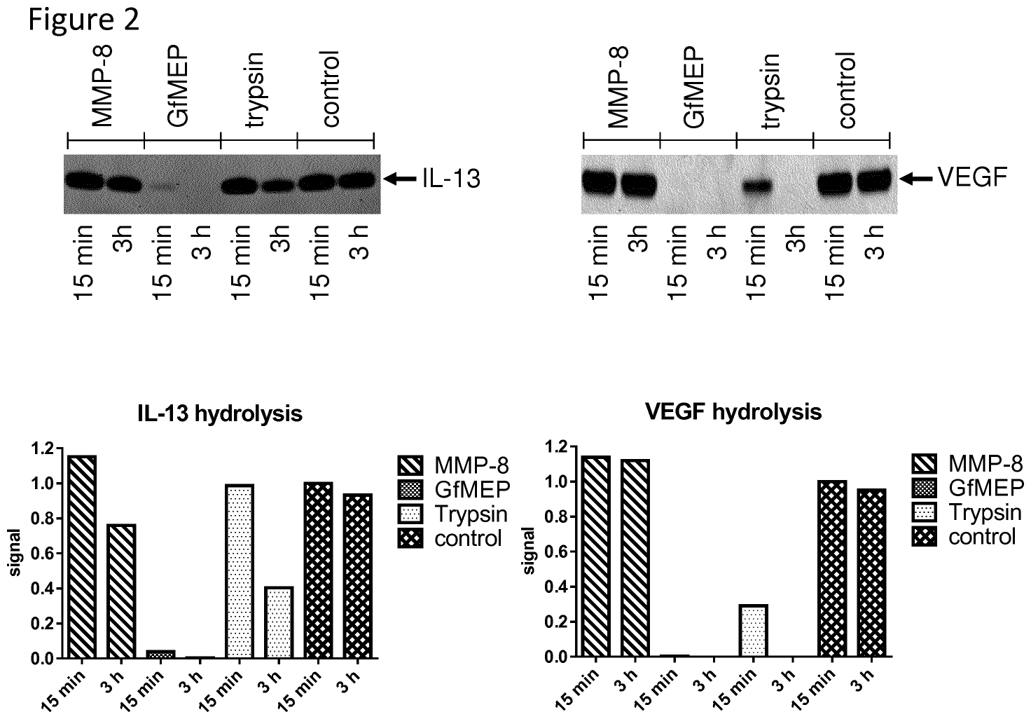

To qualitatively assess the relative efficiency of GfMEP catalysed hydrolysis of the therapeutic targets (e.g. IL-13) relative to MMP-8 and trypsin, a hydrolysis assay was performed. 2.5 μM of substrate target (e.g. IL-13) was incubated with 125 nM MMP-8, GfMEP, trypsin or blank in assay buffer (PBS, 1 mM CaCl2, 100 μM ZnCl2) at 37° C. At 15 min and 3 h, aliquots of the incubations were removed and quenched with NuPage SDS-loading buffer containing 50 mM EDTA. Samples were subjected to SDS-PAGE, followed by coomassie staining. Gels were destained, followed by imaging and quantification using a LiCor Odyssey imaging system, as shown in FIG. 2.

### Example 3—Lysine Deletion

A variant of GfMEP was designed where all lysine residues were mutated to non-lysine amino acids based on the variation observed across an alignment of related lysine-specific protease of the M35 family. Briefly, if across these M35 lysine-specific proteases the observed consensus was found to be an amino acid other than lysine at a lysine containing position in the GfMEP sequence, then that position was mutated to the consensus amino acid. For more highly conserved lysine residues where the consensus for that position was also lysine, then the next most commonly occurring amino acid was selected. Thus we selected the following mutations: K102Q, K129D, K139Q, and K148Q. One additional mutation, D145N was also selected since in wild-type GfMEP D145 appeared to make a salt bridge with the poorly conserved K148, and asparagine is the consensus residue at position 145.

For other domains (e.g. DARPINs) lysine residues were substituted similarly, with non-lysine amino acids selected from those conserved or present across an alignment of related domains.

### Example 4—Activity Mutations

The protease therapeutics disclosed herein highly potentiate the neutralising activity towards the targeted substrate, and the format can accept lower activity protease variants while maintaining potent on target activity with the benefit of reducing off-target activity and increasing the therapeutic window. One way of lowering the activity of metalloproteases it to make mutations to the active site glutamate, such as mutation to aspartate or glutamine. This strategy is applicable to metalloproteases in general.

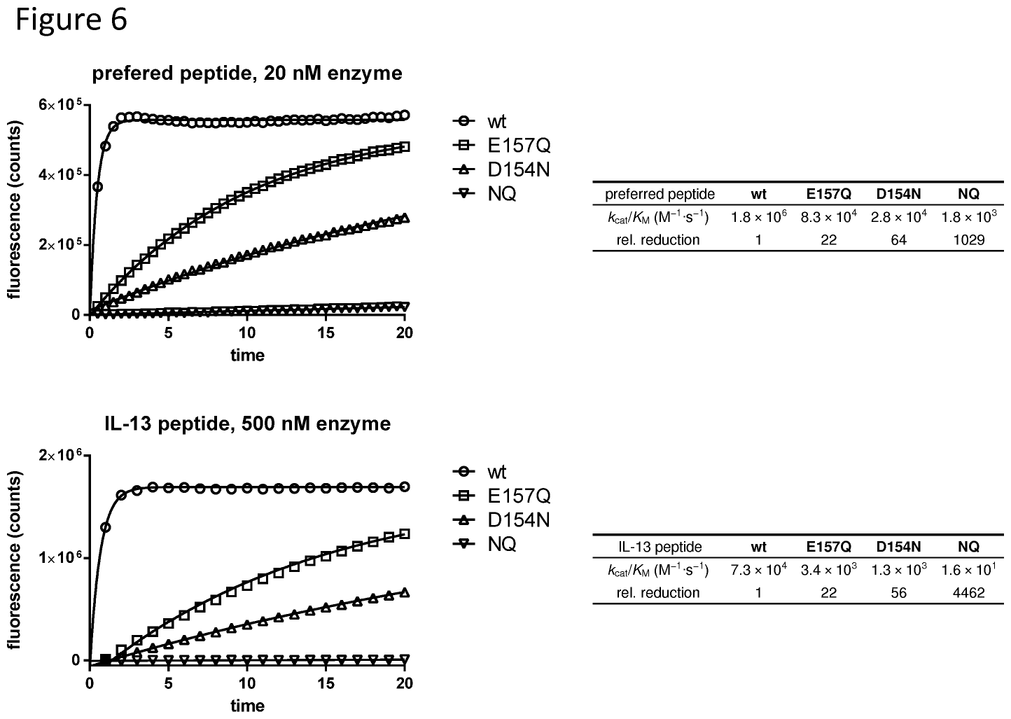

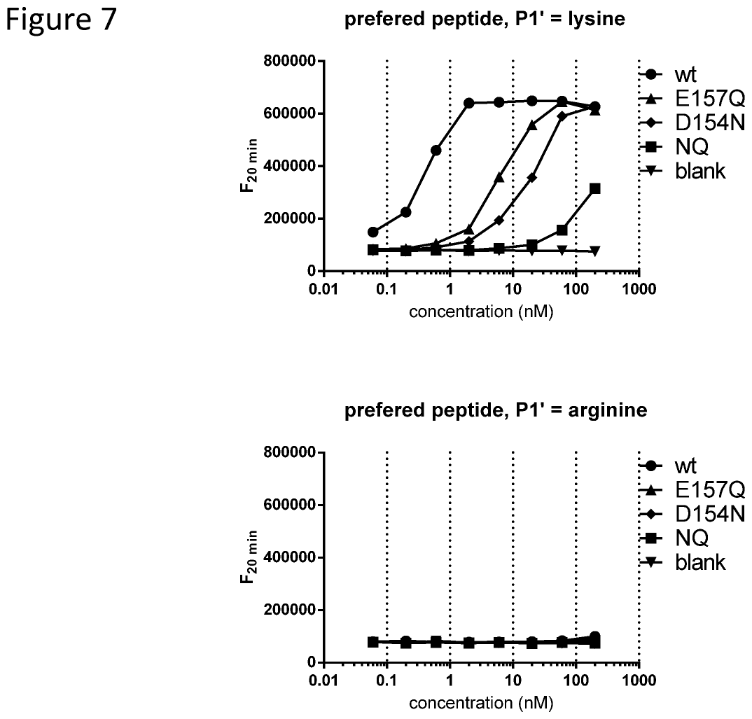

In order to find additional mutations to lower catalytic activity that would be specific to lysine-specific M35 family proteases further investigations were undertaken. To this end, the structure of GfMEP and sequence variations observed in an alignment of these M35 proteases was undertaken. From this analysis, three residues were identified that were likely important for the activity of M35 proteases: Y133, D154, and E157. These residues are invariant across the M35 family and provide key catalytic functions. Y133, via the side chain hydroxyl, acts as a hydrogen bond and proton donor to stabilise the transition state during catalysis. D154 and E157 are key residues in the S1′ substrate recognition pocket, where they define the shape and charge of the pocket to accommodate only lysine residues with high catalytic efficiency. These residues were targeted for mutation to reduce the catalytic efficiency of GfMEP and other M35 proteases. The Y133F mutation was chosen to maintain the interactions provided by the bulky hydrophobic portion while removing the contributions of the hydroxyl moiety to catalysis. The D154N and E157Q isosteric mutations were chosen to maintain the shape and size of the S1′ substrate recognition pocket while removing the stabilising charge-charge interactions that occur in this pocket between the substrate lysine and either D154, E157 or both. Constructs containing these mutations were constructed, expressed, purified and analysed as described. FIG. 6 shows the relative activity of the D154N, E157Q and the D154N/E157Q (NQ) double mutant against different substrates. FIG. 7 shows that the mutants maintain their lysine specificity.

### Example 5—Construction, Expression, Purification and Refolding

Protease therapeutics disclosed herein were constructed in a version of the pET24 expression vector, expressed in E. coli cytoplasm, purified, refolded and soluble monomeric protease therapeutics purified again as described below.

Cloning

Existing BspEI sites in the pET24d E. coli expression vector were removed by QuikChange site directed mutagenesis (Agilent Technologies). Into this modified pET24d vector, a synthetic gene encoding SEQ ID No. 14 was cloned between NcoI and Sal I sites, transformed into E. coli DH5alpha and confirmed to have the correct sequence by DNA sequencing. This created a vector with unique restriction sites for replacing any of the protease, albumin binding, or target binding domains by standard restriction subcloning. Additional sequence variants were constructed either via by QuikChange site directed mutagenesis (Agilent Technologies) or via subcloning from synthetic genes, or a combination of these techniques.

Expression, Purification and Refolding

2×800 mL of TYK in a 2 L baffled Erlenmeyer flask were inoculated with 1/200° volume of an overnight culture of E. coli BL21(DE3) colony transformed with the appropriate construct. Cultures were grown at 37° C., 180 rpm until reaching an OD600 of 0.4-0.6. 1 mM IPTG was then added to the culture flask to induce expression and the culture was continued at 37° C. for 4-5 hours. Cells were harvested by centrifugation and pellets stored at −20 until further use.

Cells were lysed using BugBuster (Merck-Millipore) with 1/1000° volume of lysonase (Merck-Millipore) according to the manufacturer's protocol but with the addition of 5 mM EDTA. The pellet was then washed three times with the same volume of pellet wash buffer (50 mM Tris,pH 8.0, 150 mM NaCl, 500 mM Urea, 1 mM EDTA). The washed pellet were stored at −20C until further use.

Washed pellets were brought to room temperature, suspended and dissolved in the same volume of pellet extraction buffer (50 mM Tris,pH 8.0, 150 mM NaCl, 8 M Urea, 1 mM EDTA). The protein solution was adjusted to 1 mg/mL in extraction buffer and 10 mM beta-mercaptoethanol. The resulting solution was left for 1 hour at room temperature before the addition of Ni-NTA sepharose 6 FF (8 mL of resin equivalent to 16 mL of slurry or equilibrated resin) that had previously been equilibrated in pellet extraction buffer.

Protein was left to bind Ni-NTA resin for 1 hour before the resin was collected in by filtration through a 10 mL gravity flow column. Resin was then washed with 100 mL wash buffer (50 mM Tris,pH 8.1, 150 mM NaCl, 8 M Urea, 20 mM imidazole) and protein eluted using 24 mL elution buffer (50 mM Tris,pH 8.1, 150 mM NaCl, 8 M Urea, 400 mM imidazole). The elution fractions were pooled and diluted in pellet extraction buffer to give a final protein concentration of 0.5 mg/mL. EDTA and beta-mercaptoethanol were then added to final concentrations of 1 mM and 10 mM, respectively. The resulting solution was then left for 1 hour at room temperature before refolding.

The protein was then refolded by rapid dilution into a 50x volume of rapidly mixing pre-chilled refolding buffer (50 mM Tris,pH 8.0, 150 mM NaCl, 1 mM EDTA, 1 mM reduced glutathione, 1 mM oxidized glutathione) and left over 2, 3 or 4 days at 4° C.

The resulting solution was purified on 5 mL prepacked Q-column and eluted in a gradient of buffer B where buffer A contains 20 mM Tris, pH 8, 1 mM EDTA; and buffer B contains 20 mM Tris, pH 8, 1 mM EDTA, 1.5 M NaCl). Fractions containing monomeric protein were then pooled and diluted 5 fold with buffer A before loading onto a prepacked Q-column (Q HP 1 mL) at 1 mL/min. Loaded protein was then eluted from the column using over a gradient buffer B.

Fractions containing monomeric protein were pooled and buffer exchanged 3 times against 50 mM Tris,pH 8.0, 150 mM NaCl, 1 mM EDTA, using Vivaspin 20 concentrators, 10000 MWCO.

### Example 6—Competition ELISA

96-well Maxisorp plates were coated with either IL-13Ralpha2 or CAT-354 (serving an IL-13Ralpha2 surrogate) at 10 μg/ml in PBS, 50 μl/well overnight at 4° C. ELISA plates were then rinsed 3× with PBS to remove unbound protein, and then blocked with 250 μl, 2% (w/v) skimmed milk powder in PBS at room temperature for 1 h.

A dilution buffer was prepared containing 0.5% skim milk, 0.1% hAlbumin, 200 μM ZnCl2 in PBS +0.05% Tween 20. This was sterile filtered and then FLA-tagged hIL-13 was added to a final concentration of 10 ng/ml. Samples of IL-13 in the presence of varying concentration of IL-13 inhibitors (protease therapeutics, antibodies) were then prepared by serial dilution of the inhibitors into the dilution buffer. Samples were then incubated at 37° C.

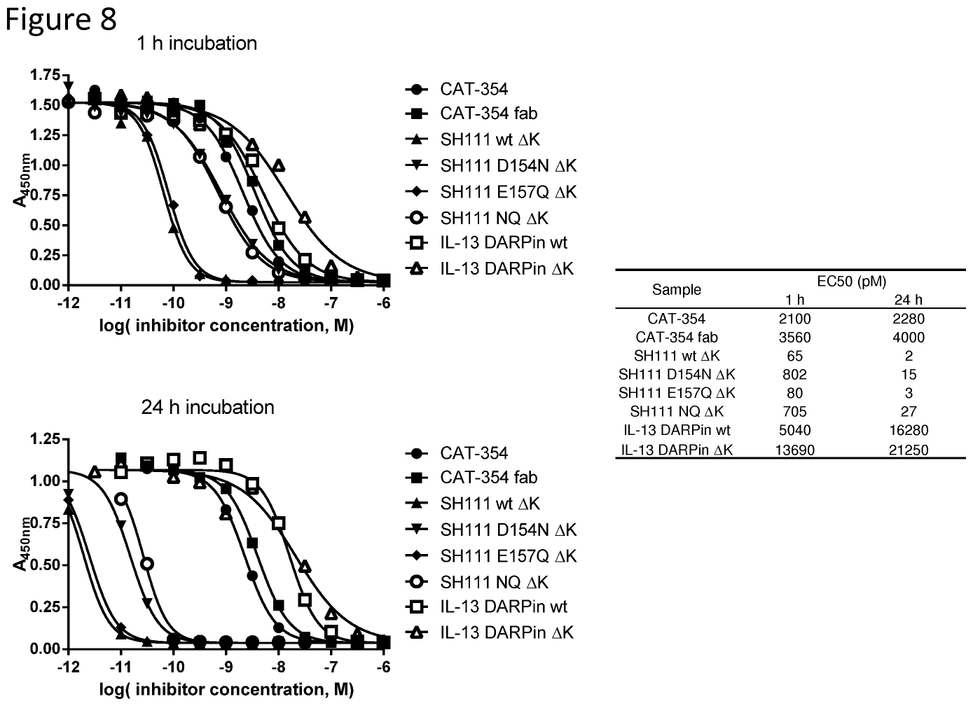

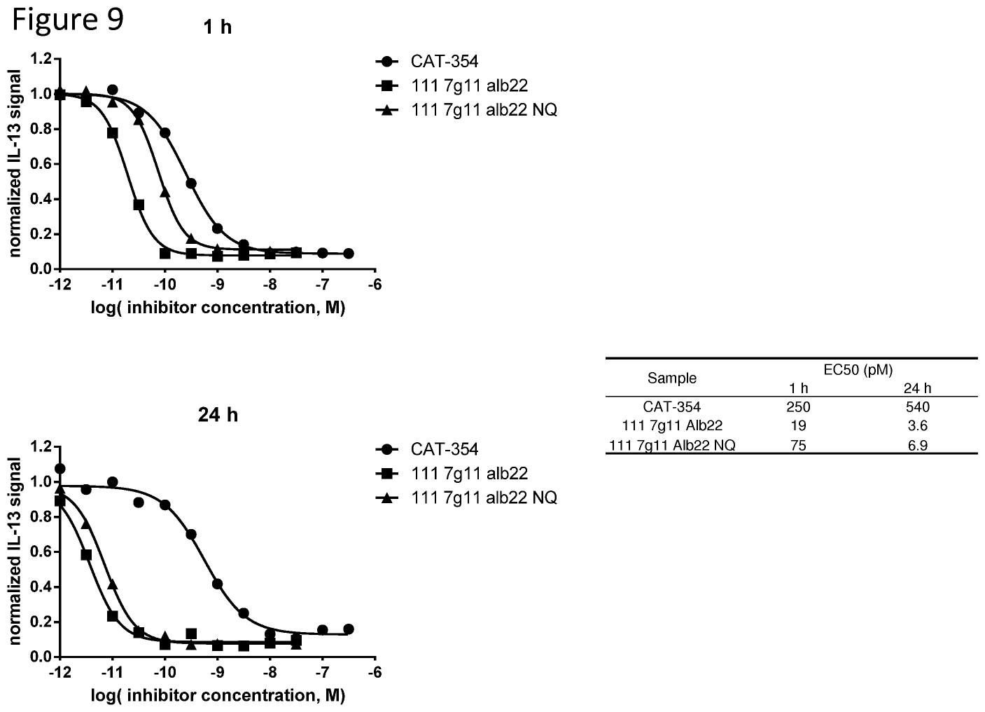

At assay time points (e.g. 1 h, 24 h) 50 μl of each of the samples were transferred to wells of the blocked ELISA plate which had been washed 3× with PBS +0.05% Tween 20 (PBST). The sample containing ELISA plates were then incubated for 1 h at room temperature. The plates were then washed 5× with PBST, followed by the addition of 50 μl/well anti-FLAG-HRP diluted 1/2000 in 0.5% (w/v) skimmed milk powder in PBST. Plates were then washed 3× with PBST, followed by 5× PBS. Plates were developed by the addition of 50 μl/well TMB substrate and quenched after 10 min by the addition of 50 μl/well 0.5 M H2SO4. Plates were read in a plate reader and the absorbance was measured at 450 nm. Results of this analysis are shown in FIG. 8. The absorbance as a function of the inhibitor concentration was plotted in GraphPad Prism and fitted to a four-parameter inhibition function to estimate EC50s. The results of this analysis are shown in FIG. 9.

### Example 7—Bioassays

Ten microliters of a 1.32 μM stock solution of each MMP construct or antibody/inhibitor control (IL13Ralpha2-Fc, CAT-354 or isotype controls) was added to 200 μL of IL-13 (Peprotech, UK) diluted to 10 ng/mL in assay media [RPMI-1640 glutamax (Invitrogen, UK), 5% heat inactivated foetal bovine serum, 1% sodium pyruvate (Sigma, UK), 1% Penicillin/Streptomycin (Invitrogen, UK), 1 mM CaCl2 and 20 μM ZnCl2 (Sigma, UK)]. Serial one in five dilutions of the construct or antibody/inhibitor controls were then made and incubated for either one or 24 hours at 37° C., 5% CO2. After incubation period 100 μL of construct/IL-13/media was added to TF1 cells set up as follows. TF1 cells (R&D Systems, UK) were washed three times in assay media and then re-suspended to a final concentration of 2×10\hat{ }5 cells/mL in the same culture media. 100 μls of cell suspension was dispensed into each well of a 96 well, flat bottomed plate and 100 μl of MMP construct or antibody/inhibitor control/IL-13/media titration was added. IL-13 or assay media alone served as positive or negative controls respectively. Cells were cultured for 3 days at 37° C., 5% CO2. After this culture period plates were pulsed with 0.2 μCi/well of tritiated thymidine (GE LifeSciences, UK) for 4 hours at 37° C., 5% CO2. Cells were then harvested onto glass fibre filter plates and dried. 50 μl of scintillant (Microscint, Perkin Elmer,UK) was dispensed onto each well of the filterplates, sealed and then thymidine incorporation determined using a liquid scintillation counter (Topcount, Perkin Elmer, UK) and expressed as counts per minute (c.p.m.).

### Example 8—Airpouch Model

On days 0 and 7 female BALB/c mice were sensitised by subcutaneous (s.c.) injection of ovablumin (10 ug) in AlOH3 or AlOH3 alone. On day 8 mice were briefly anaesthetized with isofluorane and 2.5 mL sterile air (0.25 μm filtered) was injected subcutaneously between the scapulas to create a centrally positioned air pouch. On day 11 the injection with sterile air was repeated to re-inflate the air pouch.

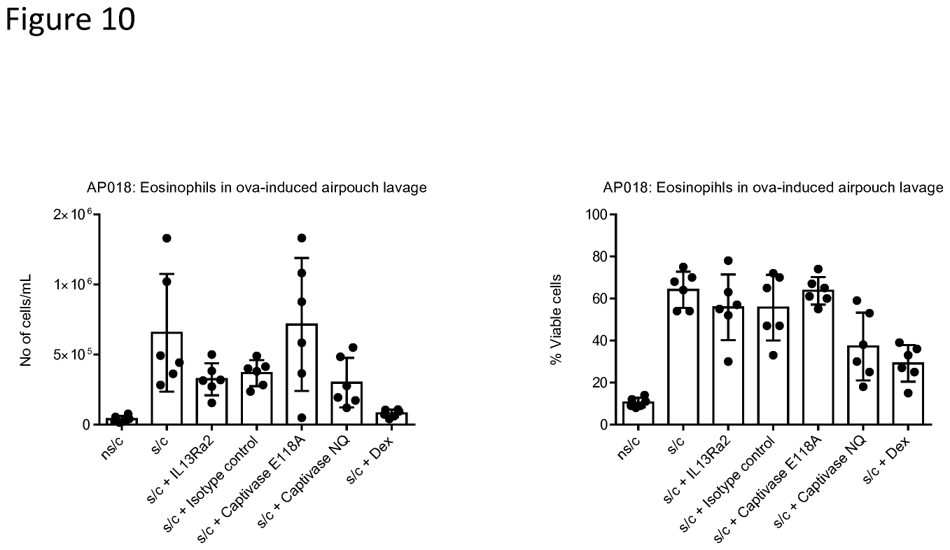

On day 14, animals were treated directly to the pouch (i.po) with protease therapeutic or PBS in 0.75% carboxymethylcellulose (CMC) 30 min before and 6 hours after induction of inflammation by i.po. Injection of ovalbumin (10 ug) in 0.75% CMC. A group of mice received dexamethasone (1.5 mg/mL) s.c instead of protease therapeutic. Twenty-four hours following induction of inflammation mice were killed and the air pouch lavaged with 1 mL heparinized PBS (5 U·mL-1). Total cells infiltrating the air pouch were counted on a MACSQuant flow cytometer. Differential cell counts were determined by Diff-Quik staining of cytospun cells. The results are shown in FIG. 10.

1. A protease therapeutic comprising a Lysine-specific metalloprotease domain conjugated to a first targeting moiety.

2. A protease therapeutic according to claim 1 wherein the Lysine-specific metalloprotease domain is not subject to inhibition by a serine protease inhibitor (SERPIN) or alpha-2-macroglubulin.

3. A protease therapeutic according to claim 1 or 2 wherein the Lysine-specific metalloprotease domain comprises a metalloendoprotease.

4. A protease therapeutic according to any one of claims 1 to 3 wherein the Lysine-specific metalloprotease domain comprises a metalloendoprotease selected from the M35 family.

5. A protease therapeutic according to any one of claims 1 to 4 wherein the Lysine-specific metalloprotease domain comprises a Grifola frondosa metalloendoprotease (GfMEP) domain.

6. A protease therapeutic according to claim 5 in which the metalloprotease domain is a non-naturally occurring mutant metalloprotease domain.

7. A protease therapeutic according to any one of claims 1 to 6 in which all protease accessible lysine residues in the metalloprotease domain have been substituted.

8. A protease therapeutic according to claim 7 in which all lysine residues in the metalloprotease protease domain have been substituted.

9. A protease therapeutic according to any one of claims 7 to 9, wherein the metalloprotease domain comprises no primary amines, except for the N-terminal amine.

10. A protease therapeutic according to any one of claims 1 to 9 wherein the active regions of the metalloprotease domain have been modified to reduce the proteolytic activity of the metalloprotease domain.

11. A protease therapeutic according to claim 10 wherein the modifications to the metalloprotease domain maintain the specificity of the metalloprotease domain.

12. A protease therapeutic according to claim 10 or 11, wherein
(i) one or more amino acid residues of the metalloendoprotease domain equivalent to residues 118, 133, 154 and 157 of SEQ ID NO. 1, are substituted; or
(ii) one or more amino acid residues selected from the group of 118, 133, 154 and 157 of SEQ ID NO. 1, are substituted.

13. A protease therapeutic according to claim 12, wherein the substitutions are selected from the group consisting of E118D, E118Q, E118N, E118S, E118A, Y133F, D154N, and E157Q.

14. A protease therapeutic according to any one of claims 5 to 13, wherein the metalloprotease domain comprises a GfMEP protease domain comprising a sequence at least 90%, at least 95%, at least 98% or at least 99% identical to SEQ ID NO: 1.

15. A protease therapeutic according to claim 14, wherein the GfMEP protease domain comprises a sequence selected from the group consisting of SEQ ID NOs: 2 to 5.

16. A protease therapeutic according to claim 15, wherein the GfMEP protease domain comprises SEQ ID NO: 2.

17. A protease therapeutic according to claim 15, wherein the GfMEP protease domain comprises SEQ ID NO. 3

18. A protease therapeutic according to claim 15, wherein the GfMEP protease domain comprises SEQ ID NO. 4

19. A protease therapeutic according to claim 15, wherein the GfMEP protease domain comprises SEQ ID NO. 5

20. A protease therapeutic according to any one of claims 15 to 19, wherein the GfMEP protease domain further comprises a substitution at position 118, such as E118D, E118Q, E118N, E118S or E118A.

21. A protease therapeutic according to claim 15, wherein the GfMEP protease domain further comprises a substitution at position 133, such as Y133F.

22. A protease therapeutic according to claim 5, wherein the GfMEP protease domain comprises the sequence of SEQ ID NO: 1.

23. A protease therapeutic according to any one of the preceding claims, wherein the first targeting moiety is selected from the group consisting of a targeting peptide, an antibody mimetic, a Tn3 scaffold, an antibody or antigen binding fragment thereof, a scFv, a Fab, a Fab′, a domain antibody, a DARPin, an aptamer and a receptor domain.

24. A protease therapeutic according to claim 23 wherein the first targeting moiety is an antibody, or antigen binding fragment thereof.

25. A protease therapeutic according to claim 23 wherein the first targeting moiety is a DARPin.

26. The protease therapeutic according to any one of the preceding claims, wherein the protease therapeutic is further conjugated to a second moiety.

27. The protease therapeutic according to claim 26, wherein the second moiety is a second targeting moiety.

28. The protease therapeutic according to claim 27, wherein second targeting moiety is selected from the group consisting of a targeting peptide, an antibody mimetic, a Tn3 scaffold, an antibody or antigen binding fragment thereof, a scFv, a Fab, a Fab′, a domain antibody, a DARPin, an aptamer and a receptor domain.

29. The protease therapeutic according to claim 27 wherein the first and second targeting moieties are directly conjugated so as to form a bispecific targeting moiety.

30. The protease therapeutic according to claim 26, wherein the second moiety is a half-life extension moiety.

31. The protease therapeutic according to claim 30, wherein the half-life extension moiety is selected from the group consisting of an albumin binding domain, albumin, a Fc region, polyethylene glycol, a XTEN fusion peptide, and a Proline/Alanine/Serine (PAS) polypeptide.

32. The protease therapeutic according to claim 31, wherein the albumin binding domain is an albumin-binding DARPin.

33. The protease therapeutic according to any preceding claim, wherein the metalloprotease domain is conjugated to the first targeting moiety via a first linker.

34. The protease therapeutic according to any one of claims 26-33, wherein the second targeting moiety is conjugated to the protease therapeutic via a second linker.

35. The protease therapeutic according to any preceding claim wherein the targeting moieties, half-life extension moieties and/or the linkers are lysine free.

36. The protease therapeutic according to any one of the preceding claims and having a sequence according to SEQ ID NO: 11.

37. The protease therapeutic according to any one of the preceding claims and having a sequence according to SEQ ID NO: 12.

38. The protease therapeutic according to any one of the preceding claims and having a sequence according SEQ ID NO: 13.

39. The protease therapeutic according to any one of the preceding claims and having a sequence according to SEQ ID NO: 14.

40. The protease therapeutic according to any one of the preceding claims and having a sequence according to SEQ ID NO: 15.

41. The protease therapeutic according to any one of the preceding claims and having a sequence according to SEQ ID NO: 16.

42. The protease therapeutic according to any one of the preceding claims and having a sequence according to SEQ ID NO: 17.

43. The protease therapeutic according to any one of the preceding claims and having a sequence according to SEQ ID NO: 18.

44. The protease therapeutic according to any one of the preceding claims, wherein the protease therapeutic is expressed as a recombinant fusion peptide or protein.

45. The protease therapeutic according to any one of the preceding claims, wherein the metalloprotease domain and targeting moieties are expressed separately and chemically conjugated.

46. The protease therapeutic according to claim 45 wherein the chemical conjugation used is solid phase chemical ligation, cysteine-maleimide conjugation, oxime conjugation, or click chemistry conjugation.

47. The protease therapeutic according to any one of the preceding claims for use in therapy.

48. The protease therapeutic according to claim 47, wherein the use comprises treatment of cancer, a respiratory condition, an inflammatory condition, a cardiovascular condition or a metabolic condition.

49. A method of treatment comprising administering a therapeutically effective amount of a protease therapeutic according to any one of the preceding claims to a patient in need of therapy.

50. The method of claim 49 wherein the patient has cancer, a respiratory condition, an inflammatory condition, a cardiovascular condition or a metabolic condition.
